(19) United States
(12) Patent Application Publication
(10) Pub. No.: US 2017/0164967 A1
(43) Pub. Date: Jun. 15, 2017

(54) Liquid Ejection Control Apparatus, Liquid Ejection System, and Control Method

(71) Applicant: Seiko Epson Corporation, Tokyo (JP)

(72) Inventor: Junichi Karasawa, Shimosuwa-machi (JP)

(73) Assignee: Seiko Epson Corporation, Tokyo (JP)

(21) Appl. No.: 15/325,480

(22) PCT Filed: Jul. 3, 2015
(86) PCT No.: PCT/JP2015/003374

§ 371 Date: Jan. 11, 2017

(30) Foreign Application Priority Data
Jul. 11, 2014 (JP) 2014-143096
Nov. 14, 2014 (JP) 2014-231468

(51) Int. Cl. A61B 17/3203; F04B 43/04; B26F 3/00

(52) U.S. Cl. CPC A61B 17/3203; B26F 3/004; F04B 43/04

(57) ABSTRACT

In a liquid ejection control apparatus, an operation unit includes an adjustment lever that is used to input an indication value of momentum or kinetic energy related to a pulsed liquid jet ejected from a liquid ejection device. A control unit includes a frequency setting portion that sets a rising frequency of a drive voltage waveform applied to a piezoelectric element and an amplitude setting portion that sets the amplitude of the drive voltage waveform, so that momentum or kinetic energy has an indication value.

### TECHNICAL FIELD

The present invention relates to a liquid ejection control apparatus and the like controlling a liquid ejection device which ejects a liquid in a pulse form by using a piezoelectric element.

### BACKGROUND ART

There is a technique of cutting a cutting target object by ejecting a liquid in a pulse form. The liquid ejected in a pulse form is a liquid jet flow which is ejected from a nozzle in a pulsating manner, and is referred to as a “pulsed liquid jet” as appropriate in the present specification.

A pulsed liquid jet may be variously applied, and, for example, PTL 1 has proposed a technique in which the pulsed liquid jet is used for surgery in a medical field. In this case, a cutting target object is a living tissue, and a liquid is physiological saline.

### CITATION LIST

### Patent Literature

PTL 1: JP-A-2005-152127

### SUMMARY OF INVENTION

### Technical Problem

As one of mechanisms generating a pulsed liquid jet, there is a mechanism using a piezoelectric element. The mechanism applies a pulsed drive voltage to a piezoelectric element so that the piezoelectric element generates instantaneous pressure in a working fluid (liquid), and thus ejects the liquid in a pulse form. Thus, the strength of the pulsed liquid jet is changed by controlling a drive voltage applied to the piezoelectric element.

Therefore, there may be a technique in which a characteristic value of a drive voltage applied to a piezoelectric element, for example, the amplitude (which is voltage amplitude and can be said to the magnitude of the drive voltage) of a drive voltage waveform is indicated by using an operation unit such as an operation dial or an operation button, and thus the strength of a pulsed liquid jet is changed.

However, it has been found that, even if the characteristic value of the drive voltage indicated by the operation unit is changed, there is a case where a cutting aspect such as a cut depth or a cut volume of a cutting target object may not be changed as intended by a user. As will be described later in detail, it has been found that, for example, even if the user changes the voltage amplitude to twice or four times, or a half or a quarter, a cut depth or a cut volume is not necessarily changed in proportion thereto. In a case where a pulsed liquid jet is used for surgery, there may be a problem in that working corresponding to an operator's operation sense is not performed.

The invention has been made in light of the above-described problems, and an object thereof is to provide a technique capable of setting the strength of a pulsed liquid jet as intended by a user so as to improve convenience.

### Solution to Problem

In order to solve the above-described problem, a first invention is directed to a liquid ejection control apparatus electrically connected to and controlling a liquid ejection device which ejects a liquid in a pulse form by using a piezoelectric element, the liquid ejection control apparatus including an operation unit that is used to input an indication value of momentum or kinetic energy related to a pulsed liquid jet ejected from the liquid ejection device; and a control unit that controls an index value related to the amplitude of a drive voltage waveform applied to the piezoelectric element and an index value of rising of the drive voltage waveform, so that the indication value is obtained.

In this case, as another invention, the liquid ejection control apparatus may be configured as a control method for a liquid ejection device which ejects a liquid in a pulse form by using a piezoelectric element, the method including inputting an indication value of momentum or kinetic energy related to a pulsed liquid jet ejected from the liquid ejection device; and controlling an index value related to the amplitude of a drive voltage waveform applied to the piezoelectric element and an index value of rising of the drive voltage waveform, so that the indication value is obtained.

A second invention is directed to a liquid ejection control apparatus electrically connected to and controlling a liquid ejection device which ejects a liquid in a pulse form by using a piezoelectric element, the liquid ejection control apparatus including an operation unit that is used to input an indication value of momentum or kinetic energy related to a pulsed liquid jet ejected from the liquid ejection device; and a control unit that controls an index value related to the amplitude of a drive voltage waveform applied to the piezoelectric element or an index value of rising of the drive voltage waveform, so that the indication value is obtained.

In this case, as another invention, the liquid ejection control apparatus may be configured as a control method for a liquid ejection device which ejects a liquid in a pulse form by using a piezoelectric element, the method including inputting an indication value of momentum or kinetic energy related to a pulsed liquid jet ejected from the liquid ejection device; and controlling an index value related to the amplitude of a drive voltage waveform applied to the piezoelectric element or an index value of rising of the drive voltage waveform, so that the indication value is obtained.

According to the first invention, the second invention and the like, if an indication value of momentum or kinetic energy related to a pulsed liquid jet ejected from the liquid ejection device is input, at least one of index values related to the amplitude and rising of a drive voltage waveform applied to the piezoelectric element is controlled so that the indication value is obtained. As will be described later, a cut depth or a cut volume is highly correlated with momentum or kinetic energy related to a pulsed liquid jet. Thus, since momentum or kinetic energy related to a pulsed liquid jet is directly indicated, it is possible to realize a cut depth or a cut volume to be suitable for a user's intention or operation sense and thus to improve convenience.

A third invention is directed to the liquid ejection control apparatus according to the first or second invention further including a display control unit that performs control of displaying the indication value of momentum or kinetic energy related to the pulsed liquid jet.

According to the third invention, it is possible to display an indication value of momentum or kinetic energy related to a pulsed liquid jet. Thus, a user can visually recognize an index indicating a strength of a desired pulsed liquid jet or the present strength thereof. Therefore, it is possible to further improve convenience.

A fourth invention is directed to the liquid ejection control apparatus according to any one of the first to third inventions, in which the liquid ejection device is controlled so that momentum of the pulsed liquid jet is equal to or more than 2 nanonewton seconds (nNs) and is equal to or less than 2 millinewton seconds (mNs), or kinetic energy of the pulsed liquid jet is equal to or more than 2 nanojoules (nJ) and is equal to or less than 200 millijoules (mJ).

According to the fourth invention, it is possible to control the liquid ejection device within a range in which the momentum of the pulsed liquid jet is equal to or more than 2 nNs and is equal to or less than 2 mNs, or the kinetic energy thereof is equal to or more than 2 nJ and is equal to or less than 200 mJ. Therefore, for example, the liquid ejection control apparatus is suitable to cut soft materials, for example, a living tissue, food, a gel material, and a resin material such as rubber or plastic.

A fifth invention is directed to the liquid ejection control apparatus according to any one of the first to fourth inventions, in which the liquid ejection device is controlled so that a living tissue is cut with the pulsed liquid jet.

According to the fifth invention, it is possible to control the strength of a pulsed liquid jet suitable for a surgery application, for example.

A sixth invention is directed to the liquid ejection control apparatus according to any one of the first to fifth inventions, in which the index value related to the rising is represented by time or a frequency related to the rising of the drive voltage waveform.

According to the sixth invention, it is possible to cause an index value related to rising to be represented by time or a frequency related to rising of a drive voltage waveform.

A seventh invention is directed to a liquid ejection system including the liquid ejection control apparatus according to any one of the first to sixth inventions; a liquid ejection device; and a liquid feeding pump device.

According to the seventh invention, it is possible to implement a liquid ejection system capable of achieving the operations and effects of the first to sixth inventions.

### BRIEF DESCRIPTION OF DRAWINGS

FIG. 1 is a diagram illustrating the entire configuration example of a liquid ejection system.

FIG. 2 is a diagram illustrating an internal structure of a liquid ejection device.

FIG. 3 illustrates diagrams illustrating a drive voltage waveform for a piezoelectric element corresponding to one cycle and a liquid flow velocity waveform in a liquid ejection opening.

FIG. 4 illustrates diagrams respectively illustrating mass flow flux Jm, momentum flow flux Jp, and energy flow flux Je.

FIG. 5 illustrates diagrams illustrating flow velocity waveforms of a main jet used in simulation for a destruction state of a cutting target object.

FIG. 6 illustrates diagrams illustrating simulation results (cut depths).

FIG. 7 illustrates diagrams illustrating simulation results (cut volumes).

FIG. 8 illustrates diagrams illustrating simulation results of flow velocity waveforms of a main jet.

FIG. 9 is a diagram illustrating a correspondence relationship among momentum P, a rising frequency, and a voltage amplitude.

FIG. 10 is a diagram illustrating a correspondence relationship among energy E, a rising frequency, and a voltage amplitude.

FIG. 11 is a block diagram illustrating a functional configuration example of a liquid ejection control apparatus in Example 1.

FIG. 12 is a diagram illustrating a data configuration example of a momentum conversion table.

FIG. 13 is a diagram illustrating another data configuration example of a momentum conversion table.

FIG. 14 is a flowchart illustrating a flow of a process performed by a control unit when a pulsed liquid jet is ejected.

FIG. 15 is a diagram illustrating a display screen example of a display unit.

FIG. 16 is a block diagram illustrating a functional configuration example of a liquid ejection control apparatus in Example 2.

FIG. 17 is a diagram illustrating a data configuration example of an energy conversion table.

FIG. 18 is a diagram illustrating another data configuration example of an energy conversion table.

FIG. 19 is a diagram illustrating a display screen example of a display unit.

### DESCRIPTION OF EMBODIMENTS

Hereinafter, a description will be made of embodiments of a liquid ejection control apparatus and a liquid ejection control method according to the invention. The invention is not limited to the embodiments described below, and embodiments to which the invention is applicable are not limited to the embodiments described below. The same portions are given the same reference numerals throughout the drawings.

Entire Configuration

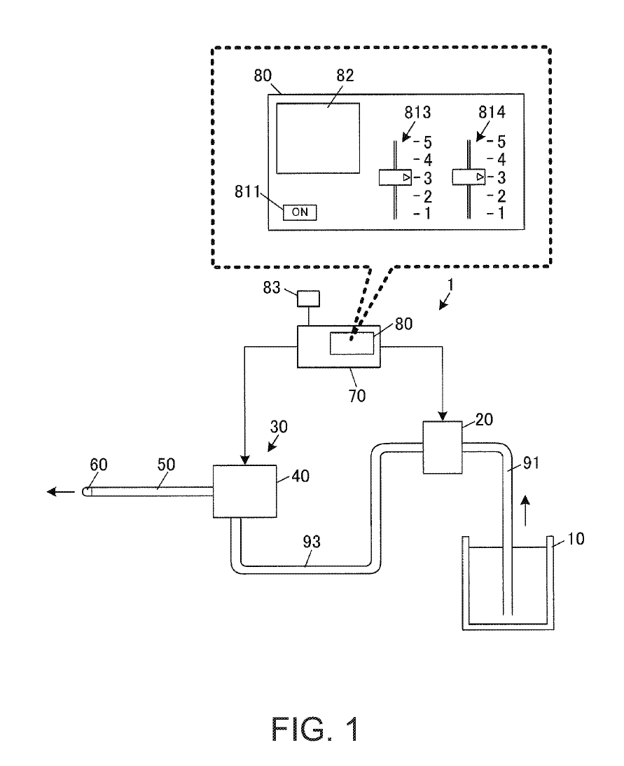

FIG. 1 is a diagram illustrating the entire configuration example of a liquid ejection system 1 in the present embodiment. The liquid ejection system 1 is used for applications such as surgery with a soft material, for example, a living tissue as a cutting target object, food processing with food as a cutting target object, processing of a gel material, and cutting processing of a resin material such as rubber or plastic, and ejects a pulsed liquid jet whose momentum is equal to or more than 2 nanonewton seconds (nNs) and is equal to or less than 2 millinewton seconds (mNs), or whose kinetic energy is equal to or more than 2 nanojoules (nJ) and is equal to or less than 200 millijoules (mJ) so as to cut a cutting target object. Hereinafter, a case will be exemplified in which the liquid ejection system 1 is used for a surgery application and performs incision, excision, or crushing (these are collectively referred to as “cutting”) of the affected part (living tissue). Momentum flow velocity and momentum in the present embodiment indicate a scalar quantity in which only an ejection direction component of a pulsed liquid jet, that is, the magnitude thereof is taken into consideration.

As illustrated in FIG. 1, the liquid ejection system 1 includes a container 10 accommodating a liquid, a liquid feeding pump 20, a liquid ejection device 30 which ejects the liquid toward a cutting target object (a living tissue in the present embodiment) in a pulse form, and a liquid ejection control apparatus 70.

In the liquid ejection system 1, the liquid ejection control apparatus 70 is provided with an operation panel 80 which is operated by an operator during surgery. The operation panel 80 is provided with a button switch 811 for switching between turning-on and turning-off of power supply; a lever switch 813 which allows lever positions in five steps, provided with scales such as “1” to “5”, to be selected for an operation of changing the moment or kinetic energy; a repetition frequency setting lever switch 814 which allow lever positions in five steps, provided with scales such as “1” to “5” for inputting a repetition frequency, to be selected; and a liquid crystal monitor 82. The liquid ejection control apparatus 70 is provided with a pedal switch 83 for switching between ejection starting and ejection stoppage of a pulsed liquid jet by the operator treading thereon.

The container 10 accommodates a liquid such as water, physiological saline, or a chemical liquid. The liquid feeding pump 20 supplies the liquid accommodated in the container 10 to a pulse flow generator 40 of the liquid ejection device 30 at predetermined pressure or a predetermined flow rate via connection tubes 91 and 93.

The liquid ejection device 30, which is a portion (handpiece) operated by the operator with hands during surgery, includes the pulse flow generator 40 which gives pulsation to the liquid supplied from the liquid feeding pump 20 so as to generate a pulse flow, and a pipe-shaped ejection tube 50, and ejects the pulse flow generated by the pulse flow generator 40 from a liquid ejection opening 61 provided at a nozzle 60 through the ejection tube 50 as a pulsed liquid jet.

Here, the pulse flow indicates a pulsative flow of the liquid which considerably and rapidly changes temporally in a flow velocity or pressure thereof. Similarly, ejecting a liquid in a pulse form indicates pulsative ejection of the liquid in which a flow velocity of the liquid passing through the nozzle considerably changes temporally. In the present embodiment, a case of ejecting a pulsed liquid jet generated by applying periodic pulsation to a steady flow is exemplified, but the invention is also applicable to intermittent and fitful ejection of a pulsed liquid jet in which ejection and non-ejection of a liquid are repeatedly performed.

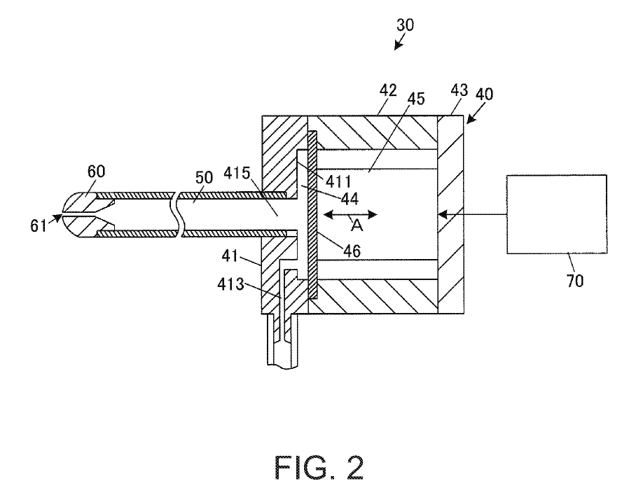

FIG. 2 is a diagram illustrating a cut surface obtained by cutting the liquid ejection device 30 along a liquid ejection direction. Vertical and horizontal scales of members or portions illustrated in FIG. 2 are different from actual ones for convenience of illustration. As illustrated in FIG. 2, the pulse flow generator 40 is configured of a piezoelectric element 45 and a diaphragm 46 which change a volume of a pressure chamber 44 and are disposed in a tubular internal space formed by a first case 41, a second case 42, and a third case 43. The respective cases 41, 42 and 43 are joined together and are thus integrally formed at surfaces facing each other.

The diaphragm 46 is a disk-shaped metal thin plate, and an outer circumferential portion thereof is interposed and fixed between the first case 41 and the second case 42. The piezoelectric element 45 is, for example, a laminated piezoelectric element, and has one end fixed to the diaphragm 46 between the diaphragm 46 and the third case 43, and the other end fixed to the third case.

The pressure chamber 44 is a space surrounded by the diaphragm 46, and a depression 411 formed on a surface facing the diaphragm 46 of the first case 41. The first case 41 is provided with an inlet channel 413 and an outlet channel 415 which communicate with the pressure chamber 44. An inner diameter of the outlet channel 415 is larger than an inner diameter of the inlet channel 413. The inlet channel 413 is connected to the connection tube 93 and introduces a liquid supplied from the liquid feeding pump 20 into the pressure chamber 44. One end of the ejection tube 50 is connected to the outlet channel 415, and thus the liquid flowing in the pressure chamber 44 is introduced into the ejection tube 50. The nozzle 60 having a liquid ejection opening 61 which has an inner diameter smaller than an inner diameter of the ejection tube 50 is inserted into the other end (front end) of the ejection tube 50.

In the liquid ejection system 1 configured in the above-described way, the liquid accommodated in the container 10 is supplied to the pulse flow generator 40 via the connection tube 93 at predetermined pressure or a predetermined flow rate by the liquid feeding pump 20 under the control of the liquid ejection control apparatus 70. On the other hand, if a drive signal is applied to the piezoelectric element 45 under the control of the liquid ejection control apparatus 70, the piezoelectric element 45 is expanded or contracted (an arrow A in FIG. 2). The drive signal applied to the piezoelectric element 45 is repeatedly applied at a predetermined repetition frequency (for example, several tens of Hz to several hundreds of Hz), and thus expansion and contraction of the piezoelectric element 45 are repeatedly performed for each cycle. Consequently, pulsation is applied to the steady flow liquid flowing in the pressure chamber 44, and thus a pulsed liquid jet is repeatedly ejected from the liquid ejection opening 61.

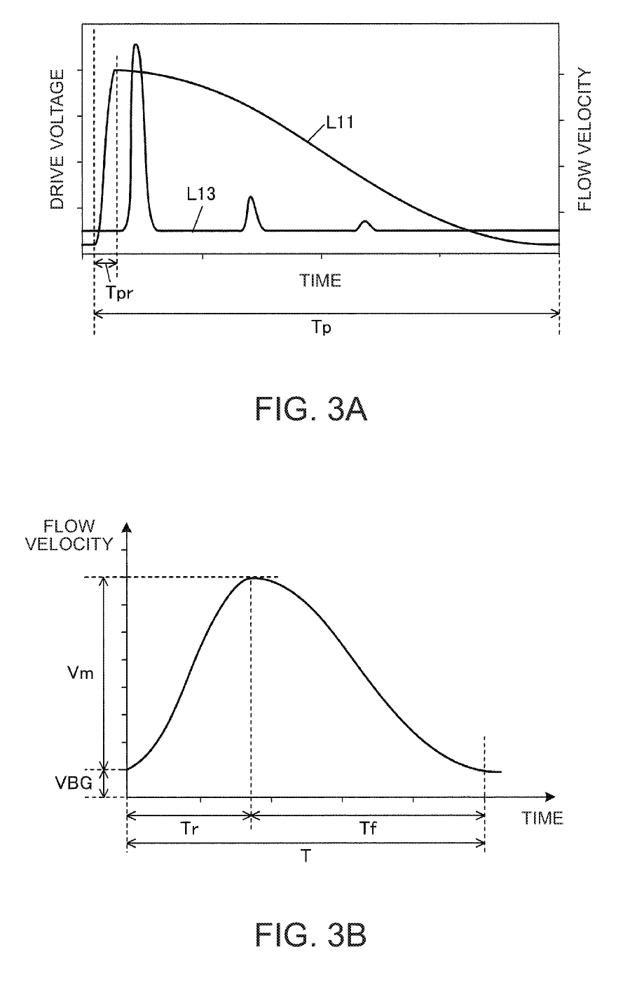

FIG. 3 (a) is a diagram illustrating an example of a drive voltage waveform L11 of a drive signal corresponding to one cycle applied to the piezoelectric element 45, and also illustrates a flow velocity waveform L13 of a liquid in the liquid ejection opening 61. Tp indicates a repetition cycle (time corresponding to one cycle of a drive voltage waveform), and an inverse number thereof is the above-described repetition frequency.

FIG. 3 (b) is a diagram obtained by extracting a main peak portion with the maximum flow velocity from among peaks of the flow velocity waveform L13 illustrated in FIG. 3 (a). The repetition cycle Tp is about 1 millisecond (ms) to 100 ms, and time (rising time) Tpr required for the drive voltage waveform to rise to the maximum voltage is about 10 microseconds (μs) to 1000 μs.

The repetition cycle Tp is set to be longer than the rising time Tpr. In a case where an inverse number of the rising time is a rising frequency, the repetition frequency is set to be lower than the rising frequency.

For example, if the piezoelectric element 45 is expanded when a positive voltage is applied thereto, the piezoelectric element 45 is rapidly expanded at the rising time Tpr, and thus the diaphragm 46 is pushed by the piezoelectric element 45 so as to be bent toward the pressure chamber 44 side. If the diaphragm 46 is bent toward the pressure chamber 44 side, the volume of the pressure chamber 44 is reduced, and thus the liquid in the pressure chamber 44 is pushed out of the pressure chamber 44. Here, the inner diameter of the outlet channel 415 is larger than the inner diameter of the inlet channel 413, fluid inertance and fluid resistance of the outlet channel 415 are less than fluid resistance of the inlet channel 413. Therefore, most of the liquid pushed out of the pressure chamber 44 due to rapid expansion of the piezoelectric element 45 is introduced into the ejection tube 50 through the outlet channel 415, and is ejected at a high speed as pulsed liquid droplets, that is, a pulsed liquid jet through the liquid ejection opening 61 having the diameter smaller than the inner diameter of the outlet channel.

The drive voltage increases to the maximum voltage, and then slowly decreases. At this time, the piezoelectric element 45 is contracted for a longer time than the rising time Tpr, and thus the diaphragm 46 is pulled to the piezoelectric element 45 so as to be bent toward the third case 43 side. If the diaphragm 46 is bent toward the third case 43 side and thus the volume of the pressure chamber 44 is increased, the liquid is introduced into the pressure chamber 44 from the inlet channel 413.

Since the liquid feeding pump 20 supplies the liquid to the pulse flow generator 40 at predetermined pressure or a predetermined flow rate, if the piezoelectric element 45 does not perform expansion and contraction operations, the liquid (steady flow) flowing in the pressure chamber 44 is introduced into the ejection tube 50 through the outlet channel 415, and is ejected from the liquid ejection opening 61. The ejected flow is a liquid flow at a constant and low speed, and may thus be regarded as a steady flow.

[Principle]

A fundamental as value indicating features of a pulsed liquid jet is the flow velocity waveform L13 of a jet corresponding to a single pulse in the liquid ejection opening 61 illustrated together with the drive voltage waveform L11 in FIG. 3(a). In the waveform a flow velocity waveform (a jet in a head wave) generated right after rising of the drive voltage and having highest peak, is to be focused. FIG. 3(b) is an enlarged view of this waveform. Other low peaks are caused by jets which are incidentally ejected since a pressure changing wave occurring in the pressure chamber 44 during expansion of the piezoelectric element 45 reflects and reciprocates in the ejection tube 50, but a destruction state of a cutting target object, that is, a cut depth or a cut volume of the cutting target object is determined by a jet in a head wave (main jet) with the highest flow velocity.

In a case where a cut depth or a cut volume of the cutting target object is to be changed by changing the strength of a pulsed liquid jet, a drive voltage waveform for the piezoelectric element 45 is controlled. There may be a method of controlling the drive voltage waveform by the operator designating a rising frequency of the drive voltage waveform or amplitude (voltage amplitude) of the drive voltage waveform as a voltage characteristic value. The rising frequency mentioned here is one of index values associated with rising of a drive voltage, and is defined as an inverse number of the rising time Tpr. For example, there may be a method in which the operator designates a rising frequency in a state in which a voltage amplitude is fixed, and designates a voltage amplitude in a state in which a rising frequency is fixed. This is because the voltage amplitude or the rising frequency (rising time Tpr) greatly influences a flow velocity waveform of the main jet. A drive voltage which is slowly decreasing after increasing to the maximum voltage does not greatly influence the flow velocity waveform of the main jet. For example, if the rising frequency is heightened, or the voltage amplitude is increased, it is considered that a cut depth and a cut volume are increased in proportion thereto.

However, it has been proved that an actually obtained cut depth or cut volume of a cutting target object may not necessarily be changed in accordance with a change in the voltage characteristic value, and thus convenience may deteriorate. For example, there is a case where the operator increases the voltage amplitude to twice, but a cut depth or a cut volume may not be increased as expected, or increases the voltage amplitude to a half, but a cut depth or a cut volume may not be reduced as expected. Thus, a situation may occur in which a cut depth or a cut volume desired by the operator is not obtained. This causes a problem of increasing surgery time.

Therefore, focusing on a flow velocity waveform of the main jet, correlations of a cut depth and a cut volume with several parameters determined by the flow velocity waveform of the main jet were examined. This is because, if a parameter highly correlated with a cut depth or a cut volume is found, the piezoelectric element 45 can be controlled with a drive voltage waveform which is optimal for achieving a cut depth or a cut volume corresponding to the operator's operation sense.

For this, first, on the basis of a flow velocity waveform v [m/s] of the main jet in the liquid ejection opening 61, mass flow flux [kg/s], momentum flow flux [N], and energy flow flux [W] of the main jet passing through the liquid ejection opening 61, were examined. The mass flow flux is mass [kg/s] per unit time of a liquid passing through the liquid ejection opening 61. The momentum flow flux is momentum [N] per unit time of a liquid passing through the liquid ejection opening 61. The energy flow flux is energy [W] per unit time of a liquid passing through the liquid ejection opening 61. The energy indicates kinetic energy, and will be hereinafter abbreviated to “energy”.

In the liquid ejection opening 61, a liquid is released to a free space, and thus pressure may be regarded to be “0”. A velocity of the liquid in a direction (a diameter direction of the liquid ejection opening 61) orthogonal to a jet ejection direction may also be regarded to be “0”. Assuming that there is no velocity distribution of a liquid in the diameter direction of the liquid ejection opening 61, mass flow flux Jm [kg/s], momentum flow flux Jp [N], and energy flow flux Je [W] of the liquid passing through the liquid ejection opening 61 may be respectively obtained according to the following Equations (1), (2) and (3). S [m²] indicates a nozzle sectional area, and ρ [kg/m³] indicates a working fluid density.

Jm=S·ρ·v (1)

Jp=S·ρ·v² (2)

Je=½·ρ·S·v³ (3)

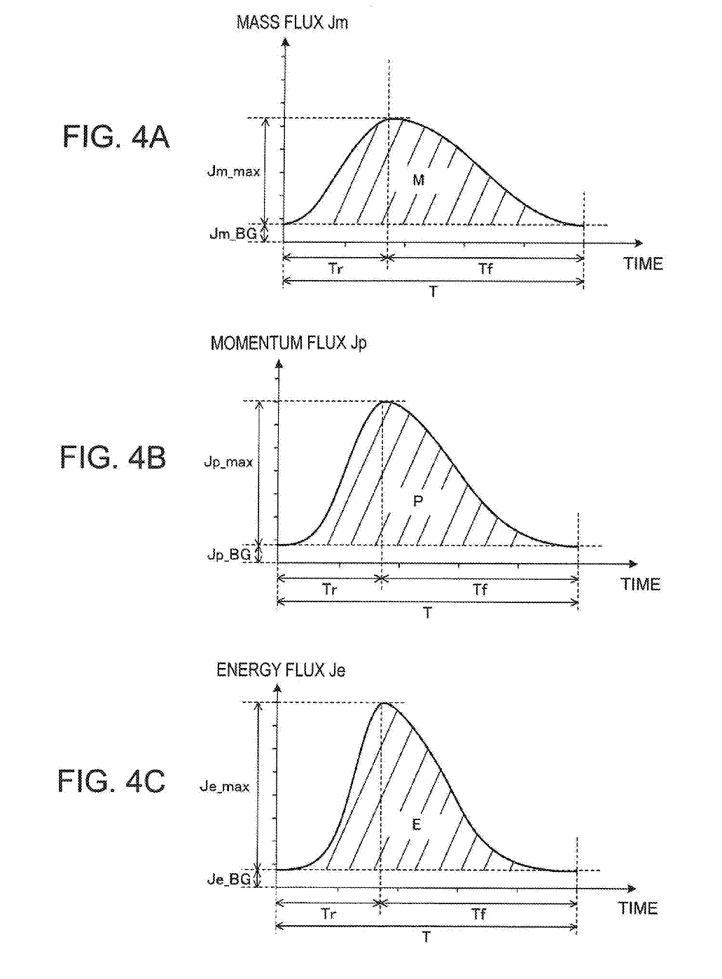

FIG. 4 illustrates diagrams respectively illustrating mass flow flux Jm (FIG. 4 (a)), momentum flow flux Jp (FIG. 4 (b)), and energy flow flux Je (FIG. 4 (c)) obtained on the basis of the flow velocity waveform of the main jet illustrated in FIG. 3 (b). If each of the mass flow flux Jm, the momentum flow flux Jp, and the energy flow flux Je is integrated over time (duration) T from rising to falling of the flow velocity waveform of the main jet, mass, momentum, and energy of a liquid ejected from the liquid ejection opening 61 as the main jet can be obtained.

The values of mass flow flux Jm, the momentum flow flux Jp, the energy flow flux Je, the mass, the momentum, and the energy calculated in the above-described way may determine a cut depth and a cut volume related to a jet corresponding to a single pulse. However, each of the above physical quantities includes a quantity corresponding to a steady flow, and it is noted that a value thereof to be obtained is a value by subtracting an attribution of the steady flow.

Therefore, regarding the mass flow flux Jm illustrated in FIG. 4(a), two parameters are defined, such as the maximum mass flow flux Jm_max [kg/s] obtained by subtracting mass flow flux Jm_BG [kg/s] of a steady flow from a peak value (maximum value) of the mass flow flux Jm, and outflow mass M [kg], hatched in FIG. 4(a), obtained by excluding an amount corresponding to the steady flow from mass of a liquid flowing out of the liquid ejection opening 61 as the main jet. The outflow mass M is expressed by the following Equation (4).

M=∫(Jm−Jm_BG)dt (4)

Regarding the momentum flow flux Jp illustrated in FIG. 4 (b), two parameters are defined, such as the maximum momentum flow flux Jp_max [N] obtained by subtracting momentum flow flux Jp_BG [N] of a steady flow from a peak value (maximum value) of the momentum flow flux Jp, and momentum P [Ns], hatched in FIG. 4(b), obtained by excluding an amount corresponding to the steady flow from momentum of a liquid flowing out of the liquid ejection opening 61 as the main jet. The momentum P is expressed by the following Equation (5).

P=∫(Jp−Jp_BG)dt (5)

Regarding the energy flow flux Je illustrated in FIG. 4 (c), two parameters are defined, such as the maximum energy flow flux Je_max [W] obtained by subtracting energy flow flux Je_BG [W] of a steady flow from a peak value (maximum value) of the energy flow flux Je, and energy E [J], hatched in FIG. 4 (c), obtained by excluding an amount corresponding to the steady flow from energy of a liquid flowing out of the liquid ejection opening 61 as the main jet. The energy E is expressed by the following Equation (6).

E=∫(Je−Je_BG)dt (6)

Here, the integration section in each of the above Equations (4), (5) and (6) is time (duration) T from rising to falling of the main jet in the flow velocity waveform.

By using numerical value simulation, to what extent each of the six parameters such as the maximum mass flow flux Jm_max, the outflow mass M, the maximum momentum flow flux Jp_max, the momentum P, the maximum energy flow flux Je_max, and the energy E is correlated with a cut depth and a cut volume was examined.

Here, a pulsed liquid jet is a fluid, and a cutting target object is a soft elastic body. Therefore, in order to perform simulation for a destruction behavior of the cutting target object using the pulsed liquid jet, an appropriate destruction threshold value is set on the soft elastic body side, and then so-called interaction analysis (fluid structure interaction (FSI) analysis) of the fluid and a structure (here, the soft elastic body) is required to be performed. Computation methods in simulation may include a finite element method (FEM), a method using a particle method whose representative is a smoothed particle hydrodynamics (SPH), and a method of combining the finite element method with the particle method. An applied method is not particularly limited. Thus, although not described in detail, an optimal method was selected by taking into consideration stability of an analysis result, computation time, and the like, and the simulation was performed.

When the simulation was performed, a fluid density=1 g/cm³, a diameter of the liquid ejection opening 61=0.15 mm, and a standoff distance (a distance from the liquid ejection opening 61 to a surface of the cutting target object)=0.5 mm were set. Assuming that the cutting target object was a soft elastic body having a flat surface, a Mooney-Rivlin super-elastic body having a density of 1 g/cm³ and an elastic modulus of about 9 kPa (about 3 kPa in terms of shear modulus) in terms of Young's modulus was used as a physical model thereof. Equivalent deviation strain=0.7 was used in the destruction threshold value.

Regarding flow velocity waveforms of the main jet, various flow velocity waveforms of the main jet were assumed, and a total of flow velocity waveforms of 27 types were prepared by changing amplitude (the maximum value of flow flux) of three types in a range of 12 m/s to 76 m/s and changing duration of three types in a range of 63 μs to 200 μs, with respect to each of waveforms of three types such as a sine wave, a triangular wave, and a rectangular wave. A flow velocity of a steady flow was 1 m/s.

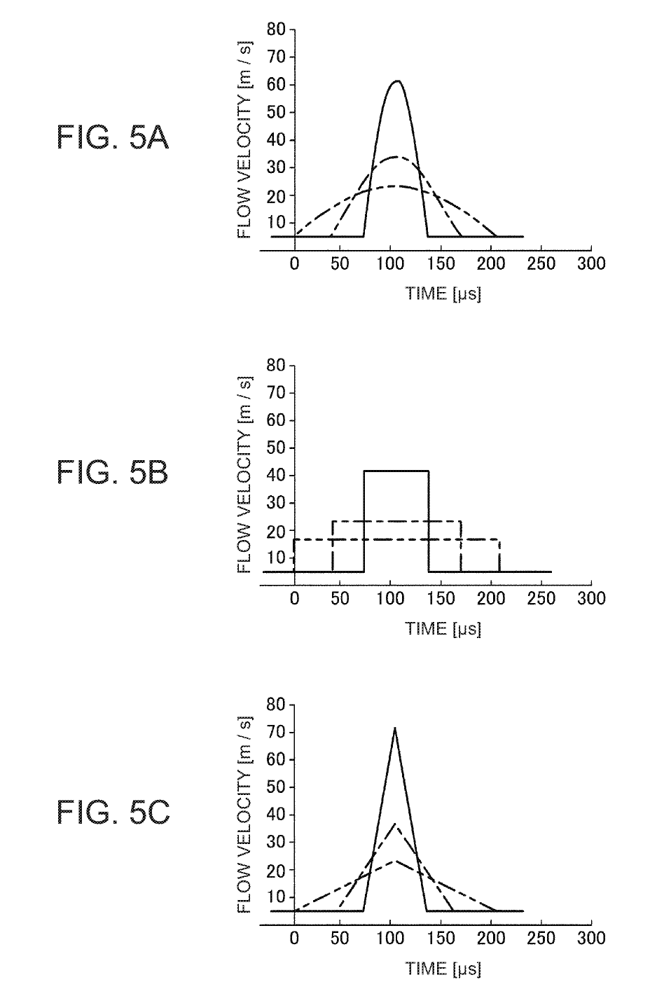

FIG. 5 illustrates a sine wave (FIG. 5(a)), a rectangular wave (FIG. 5(b)), and a triangular wave (FIG. 5(c)) provided as flow velocity waveforms of the main jet in the simulation, in which a solid line indicates a case where the duration is 63 μs, a dot chain line indicates a case where the duration is 125 μs, and a two-dot chain line indicates a case where the duration is 200 μs. The prepared waveforms were provided as flow velocity waveforms of the main jet so that pulsed liquid jets were generated, and the simulation for a destruction behavior of the soft elastic body when the pulsed liquid jets were ejected onto the soft elastic body was performed.

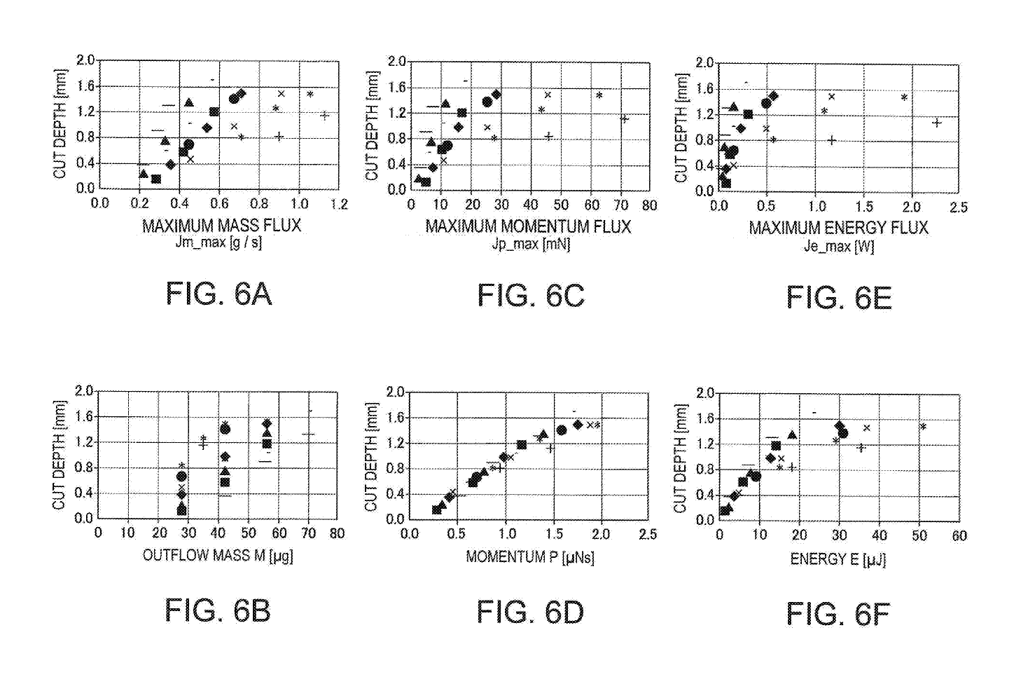

FIG. 6 illustrates diagrams respectively plotting simulation results in a case where a longitudinal axis expresses a cut depth of a cutting target object, and a transverse axis expresses the maximum mass flow flux Jm_max (FIG. 6(a)), the outflow mass M (FIG. 6(b)), the maximum momentum flow flux Jp_max (FIG. 6(c)), the momentum P (FIG. 6(d)), the maximum energy flow flux Je_max (FIG. 6(e)), and the energy E (FIG. 6(f)). In FIG. 6, a simulation result obtained when a sine wave with the duration of 63 μs is provided as a flow velocity waveform of the main jet is indicated by a plot of “*”; a simulation result obtained when a sine wave with the duration of 125 μs is provided as a flow velocity waveform of the main jet is indicated by a plot of “♦”; and a simulation result obtained when a sine wave with the duration of 200 μs is provided as a flow velocity waveform of the main jet is indicated by a plot of “−”. In addition, a simulation result obtained when a triangular wave with the duration of 63 μs is provided as a flow velocity waveform of the main jet is indicated by a plot of “+”; a simulation result obtained when a triangular wave with the duration of 125 μs is provided as a flow velocity waveform of the main jet is indicated by a plot of “X”; and a simulation result obtained when a triangular wave with the duration of 200 μs is provided as a flow velocity waveform of the main jet is indicated by a plot of a square shape displayed black. Further, a simulation result obtained when a rectangular wave with the duration of 63 μs is provided as a flow velocity waveform of the main jet is indicated by a plot of “”; a simulation result obtained when a rectangular wave with the duration of 125 μs is provided as a flow velocity waveform of the main jet is indicated by a plot of a triangular shape displayed black; and a simulation result obtained when a rectangular wave with the duration of 200 μs is provided as a flow velocity waveform of the main jet is indicated by a plot of “-”.

As illustrated in FIGS. 6(a), (c) and (e), the relationship between each of the three parameters such as the maximum mass flow flux Jm_max, the maximum momentum flow flux Jp_max, and the maximum energy flow flux Je_max, and the cut depth greatly varies depending on the shape of the waveform provided as a flow velocity waveform of the main jet, and thus it was found that a mutual correlation is low. Especially, this suggests that the mass flow flux has a value proportional to a flow velocity, and thus a cut depth is not defined by only the maximum flow velocity of the main jet.

Next, regarding the relationship between each of the three parameters such as the outflow mass M, the momentum P, and the energy E, illustrated in FIGS. 6(b), (d), and 6(f), and the cut depth, the relationship between the outflow mass M and the cut depth greatly varies depending on the shape of the waveform provided as a flow velocity waveform of the main jet, and thus a mutual correlation is low. In contrast, in the relationship with the momentum P or the energy E, a variation due to the shape of the provided waveform is small, and the respective plots are substantially distributed on the same curve. Of the momentum P and the energy E, the momentum P less varies. Therefore, it can be said that the cut depth has a high correlation with the momentum P or the energy E, and is highly correlated with, especially, the momentum P.

Here, the simulation was performed in a case where the diameter of the liquid ejection opening was 0.15 mm, and a standoff distance was 0.5 mm, but simulation was performed for other liquid ejection opening diameters or standoff distances, and it was found that a quantitative tendency that the cut depth is highly correlated with the momentum P or the energy E does not greatly change.

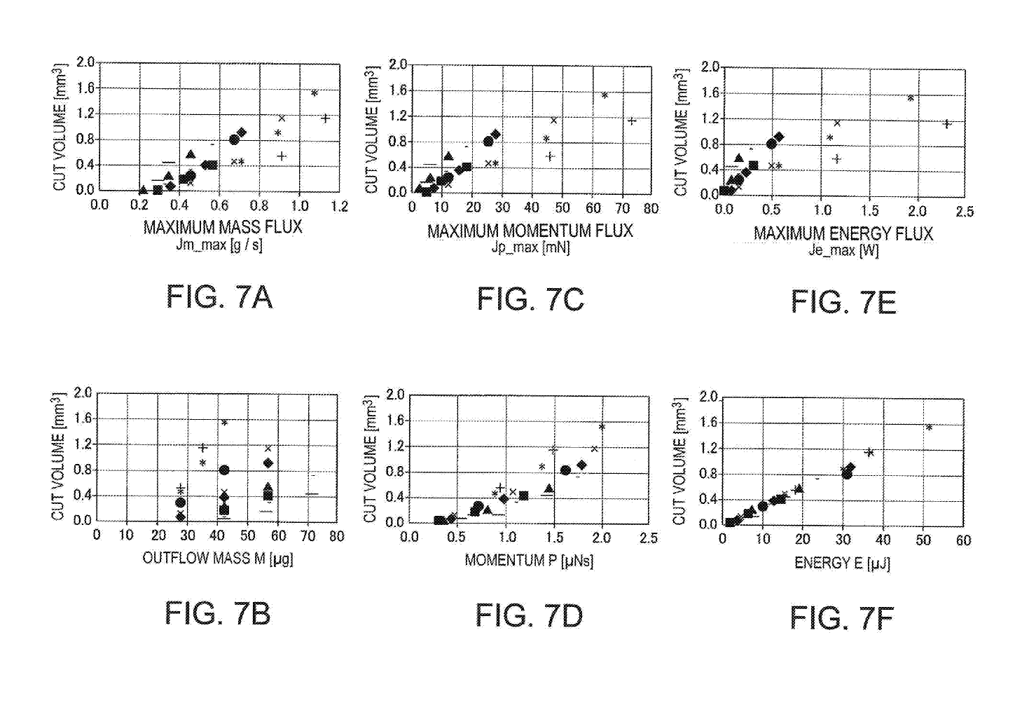

FIG. 7 illustrates diagrams respectively plotting simulation results in a case where a longitudinal axis expresses a cut volume of a cutting target object, and a transverse axis expresses the maximum mass flow flux Jm_max (FIG. 7(a)), the outflow mass M (FIG. 7(b)), the maximum momentum flow flux Jp_max (FIG. 7(c)), the momentum P (FIG. 7(d)), the maximum energy flow flux Je_max (FIG. 7(e)), and the energy E (FIG. 7(f)). Relationships between waveforms provided as a flow velocity waveform of the main jet and the types of plots are the same as in FIG. 6.

As illustrated in FIGS. 7(a), (c) and (e), the relationship between each of the three parameters such as the maximum mass flow flux Jm_max, the maximum momentum flow flux Jp_max, and the maximum energy flow flux Je_max, and the cut volume varies depending on the shape of the waveform provided as a flow velocity waveform of the main jet although not as much as the relationship with the cut depth, and thus it is considered that a mutual correlation is low.

Next, regarding the relationship between each of the three parameters such as the outflow mass M, the momentum P, and the energy E, illustrated in FIGS. 7(b), (d) and (f), and the cut volume, the relationship between the outflow mass M and the cut volume greatly varies depending on the shape of the waveform provided as a flow velocity waveform of the main jet in the same manner as in the cut depth, and thus a mutual correlation is low. In contrast, in the relationship with the momentum P or the energy E, a variation due to the shape of the provided waveform is small in the same manner as in the cut depth, and the respective plots are substantially distributed on the same line. The energy E less varies than the momentum. P. Therefore, it can be said that the cut volume has a high correlation with the momentum P or the energy E, and is highly correlated with, especially, the energy E.

Here, the simulation was performed in a case where the diameter of the liquid ejection opening was 0.15 mm, and a standoff distance was 0.5 mm, but simulation was performed for other liquid ejection opening diameters or standoff distances, and it was found that a quantitative tendency that the cut volume is highly correlated with the momentum P or the energy E does not greatly change.

In the present embodiment, on the basis of the above examination results, simulation for representative drive voltage waveforms which are actually applied to the piezoelectric element 45 is performed in advance, and thus correspondence relationships among the momentum P, the energy E, rising frequencies, and voltage amplitudes are acquired. During surgery, corresponding rising frequency and voltage amplitude are specified in response to an operation of changing the momentum P or the energy E by an operator, and driving of the piezoelectric element 45 is controlled.

First, the flow velocity waveform of the main jet is obtained through simulation by providing a drive voltage waveform in which the rising frequency is changed in steps in a state in which the voltage amplitude is fixed. Similarly, the flow velocity waveform of the main jet is obtained through simulation by providing a drive voltage waveform in which the voltage amplitude is changed in steps in a state in which the rising frequency is fixed. The simulation may be performed, for example, by using numerical value simulation which is based on a model replacing a channel system of the liquid ejection device with fluid (channel) resistance, fluid inertance, fluid compliance, or the like, and which uses an equivalent circuit method. Alternatively, fluid simulation may be used by using a finite element method (FEM), a finite volume method (FVM), or the like.

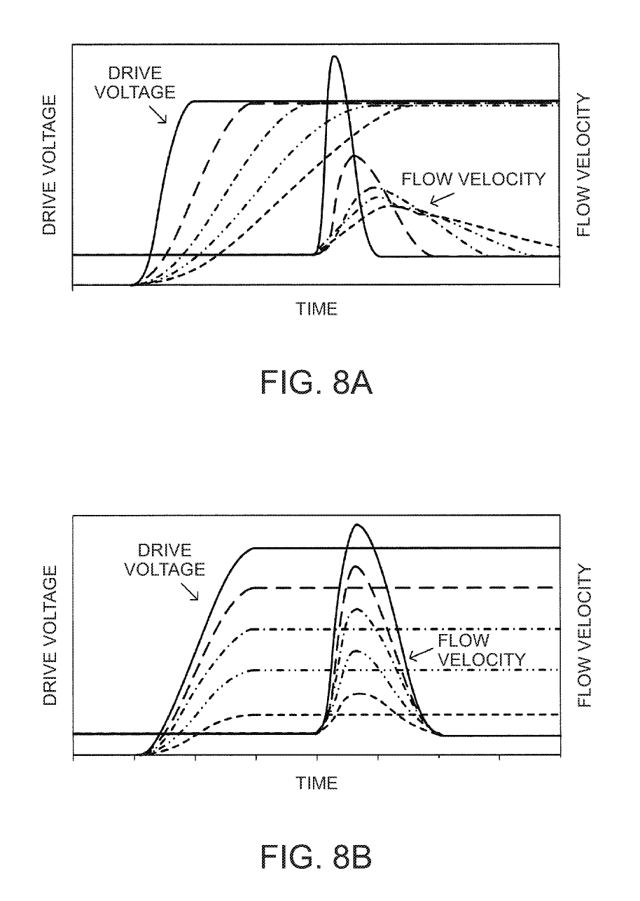

FIG. 8(a) is a diagram illustrating simulation results of the flow velocity waveform of the main jet in a case of changing the rising frequency. As illustrated in FIG. 8(a), if the rising frequency is low (the rising time Tpr is long), in the flow velocity waveform of the main jet, a rising timing does not vary, and the duration is lengthened, and thus the amplitude (the maximum value of the flow velocity) thereof is also reduced. FIG. 8 (b) is a diagram illustrating simulation results of the flow velocity waveform of the main jet in a case of changing the voltage amplitude. As illustrated in FIG. 8 (b), if the voltage amplitude is reduced, in the flow velocity waveform of the main jet, the duration during rising is maintained unlike in the cases where the rising frequency is reduced, and the waveform amplitude (the maximum value of the flow velocity) is reduced.

Next, the momentum P and the energy E are obtained for each of the obtained flow velocity waveforms of the main jet.

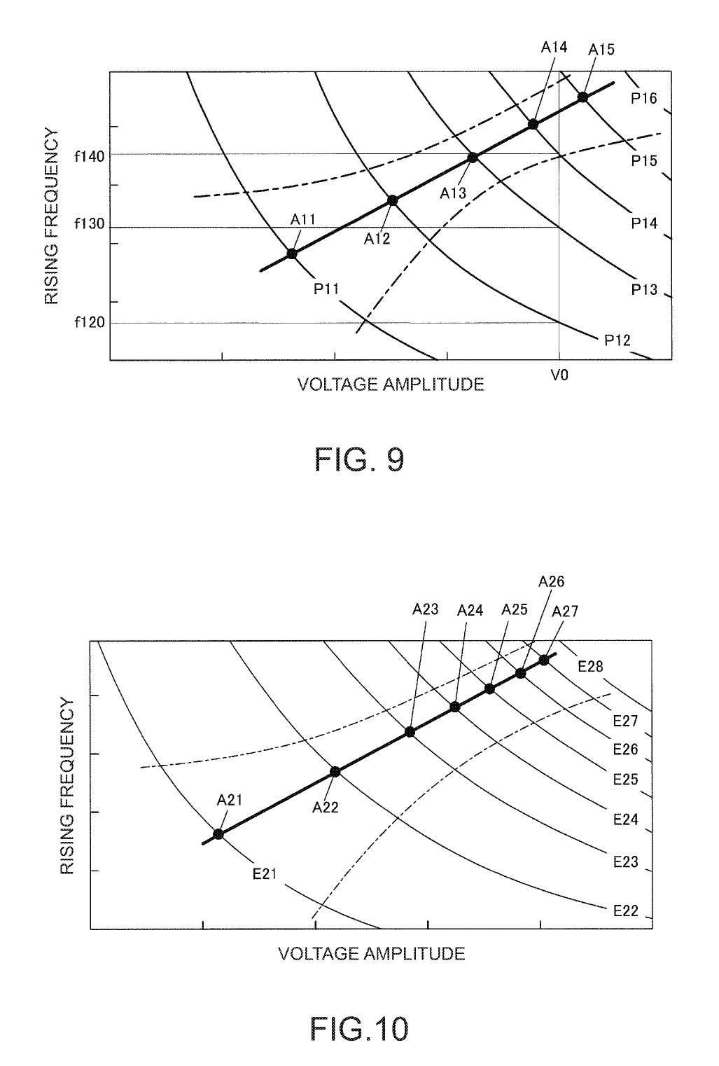

FIG. 9 is a diagram illustrating correspondence relationships among the momentum. P, the rising frequency, and the voltage amplitude for each of the obtained flow velocity waveform of the main jet. FIG. 9 is obtained by plotting the obtained momentum P in a coordinate space in which a longitudinal axis expresses the rising frequency, and a transverse axis expresses the voltage amplitude, and drawing contour lines regarding the momentum P. The respective contour lines are low on the lower left side in FIG. 9, and increase by a predetermined amount toward the upper right side.

FIG. 10 is a diagram illustrating correspondence relationships among the energy E, the rising frequency, and the voltage amplitude for each of the obtained flow velocity waveform of the main jet. Also in a case of the energy E, FIG. 10 is obtained by plotting the obtained energy E in a coordinate space in which a longitudinal axis expresses the rising frequency, and a transverse axis expresses the voltage amplitude, and drawing contour lines regarding the energy E. The respective contour lines are low on the lower left side in FIG. 10, and increase by a predetermined amount toward the upper right side.

Here, it is noted that gaps between the contour lines are not the same as each other in either case of the momentum P and the energy E, and the momentum P or the energy E does not linearly change in the coordinate axis direction. For example, in the correspondence relationships among the momentum P, the rising frequency, and the voltage amplitude illustrated in FIG. 9, a case is assumed that the voltage amplitude is fixed (to V0, for example), a drive voltage waveform for the piezoelectric element 45 is controlled by changing the rising frequency. In a case where an amount of the momentum P to be changed is to be constant, a frequency change between the rising frequencies f120 and f130 is necessary between the momentum indication values P12 and P13, and a frequency change between the rising frequencies f130 and f140 is necessary between the momentum indication values P13 and P14. However, a frequency gap between the rising frequencies f120 and f130 is different from a frequency gap between the rising frequencies f130 and f140. This phenomenon notably appears as the momentum P increases. Therefore, in a case of performing an operation of changing the rising frequency by a predetermined amount in a state in which the voltage amplitude is fixed, the momentum P does not changed as expected, and thus it can be said that a situation may occur in which a cut depth or a cut volume is not changed as intended or perceived by an operator. This may also be same for a case of performing an operation of changing the voltage amplitude by a predetermined amount in a state in which the rising frequency is fixed. This is also the same for the energy E.

Therefore, in the present embodiment, an operation performed by the operator during surgery is an operation of changing the momentum. P or the energy E using the lever switch 813. In a case of changing the momentum P, a reference line is defined in the coordinate space illustrated in FIG. 9, and rising frequencies and voltage amplitudes at respective intersections at which the reference line intersects the respective contour lines of the momentum P are acquired and are generated as a data table. For example, in a case where the straight line indicated by the bold line in FIG. 9 is used as a reference line, a data table is created in which rising frequencies and voltage amplitudes at intersections A11, A12, . . . with the respective contour lines are correlated with the momentum P of corresponding contour lines. The momentum indication values P11, P12, . . . of the respective contour lines are respectively allocated to lever positions of the lever switch 813 as indication values of the momentum P (momentum indication values). Consequently, an amount of the momentum P to be changed when the lever switch 813 is moved by one scale can be made constant.

On the other hand, in a case of changing the energy E, a reference line is defined in the coordinate space illustrated in FIG. 10, and rising frequencies and voltage amplitudes at respective intersections at which the reference line intersects the respective contour lines of the energy E are acquired and are generated as a data table. For example, in a case where the straight line indicated by the bold line in FIG. 10 is used as a reference line, a data table is created in which rising frequencies and voltage amplitudes at intersections A21, A22, . . . with the respective contour lines are correlated with the energy E of corresponding contour lines. The momentum indication values E21, E22, . . . of the respective contour lines are respectively allocated to lever positions of the lever switch 813 as indication values of the energy E (energy indication values). Consequently, an amount of the energy E to be changed when the lever switch 813 is moved by one scale can be made constant.

A reference line may not necessarily be a straight line. For example, the curve or the like indicated by the dot chain line in FIGS. 9 and 10 may be used as a reference line.

Hereinafter, as Examples of the present embodiment, a description will be made of a case (Example 1) where the operator controls a drive voltage waveform for the piezoelectric element 45 by performing an operation of changing the momentum P by using the lever switch 813, and a case (Example 2) in which the operator controls a drive voltage waveform for the piezoelectric element 45 by performing an operation of changing the energy E by using the lever switch 813.

### Example 1

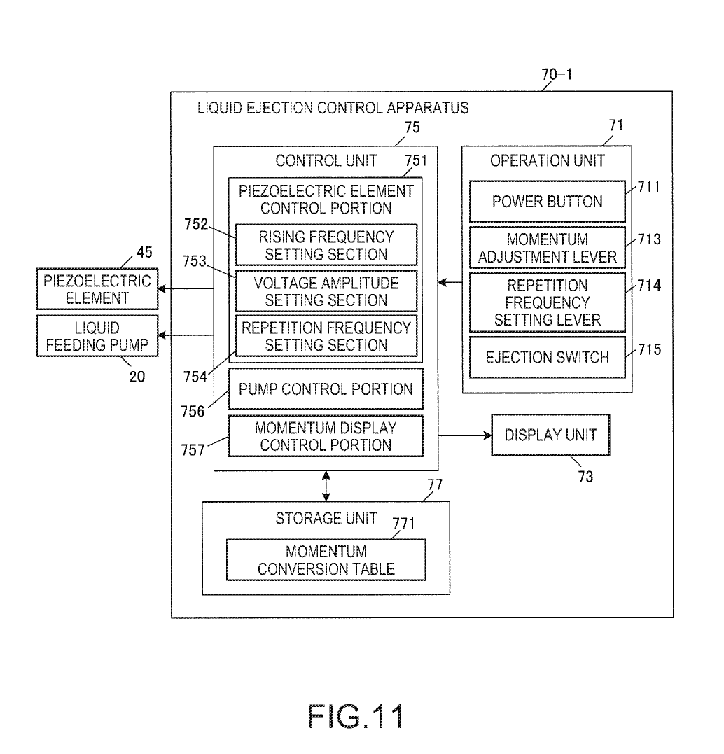

First, Example 1 will be described. FIG. 11 is a block diagram illustrating a functional configuration example of a liquid ejection control apparatus in Example 1. As illustrated in FIG. 11, a liquid ejection control apparatus 70-1 includes an operation unit 71, a display unit 73, a control unit 75, and a storage unit 77.

The operation unit 71 is implemented by various switches such as a button switch, a lever switch, a dial switch, and a pedal switch, and an input device such as a touch panel, a track pad, and a mouse, and outputs an operation signal corresponding to an input operation to the control unit 75. The operation unit 71 includes a power button 711 implemented by the button switch 811 illustrated in FIG. 1, a momentum adjustment lever 713 implemented by the lever switch 813 illustrated in FIG. 1, a repetition frequency setting lever 714 implemented by the lever switch 814 illustrated in FIG. 1, and an ejection switch 715 implemented by the pedal switch 83 illustrated in FIG. 1.

The momentum adjustment lever 713 is used to input a momentum indication value. The operator operates the momentum adjustment lever 713, that is, the lever switch 813 illustrated in FIG. 1, so as to select the lever positions with scales such as “1” to “5”, and thus performs an operation of changing the momentum. P in five steps. The momentum indication values are allocated at the respective lever positions so that the momentum P is increased by a predetermined level in proportion to a numerical value of a corresponding scale. The number of steps of the lever positions is not limited to five steps, and may be set as appropriate, for example, three steps such as “large”, “intermediate”, and “small”.

The repetition frequency setting lever 714 is used to set a repetition frequency. The operator operates the lever switch 814 illustrated in FIG. 1 so as to select lever positions with scales such as “1” to “5”, and thus performs an operation of changing a repetition frequency (for example, several tens to several hundreds of Hz) of a drive voltage waveform which is repeatedly applied to the piezoelectric element 45, in five steps. Repetition frequencies corresponding to numerical values of the scales are allocated to the respective lever positions. The number of steps of the lever positions is not limited to five steps, and may be set as appropriate.

The display unit 73 is implemented by a display device such as a liquid crystal display (LCD) or an electroluminescence (EL) display, and displays various screens such as a setting screen on the basis of display signals input from the control unit 75. The display unit 73 corresponds to, for example, the liquid crystal monitor 82 illustrated in FIG. 1.

The control unit 75 is implemented by a microprocessor such as a central processing unit (CPU) or a digital signal processor (DSP), and a control device and a calculation device such as an application specific integrated circuit (ASIC), and generally controls the respective portions of the liquid ejection system 1. The control unit 75 includes a piezoelectric element control portion 751, a pump control portion 756, and a momentum display control portion 757. The respective portions constituting the control unit 75 may be formed of hardware such as a dedicated module circuit.

The piezoelectric element control portion 751 includes a rising frequency setting section 752 which sets a rising frequency according to a lever position of the momentum adjustment lever 713, a voltage amplitude setting section 753 which sets a voltage amplitude according to a lever position of the momentum adjustment lever 713, and a repetition frequency setting section 754 which sets a repetition frequency according to a lever position of the repetition frequency setting lever 714. The piezoelectric element control portion 751 generates a drive voltage waveform, applies a drive signal having the generated waveform to the piezoelectric element 45, and, at this time, generates the drive voltage waveform according to a rising frequency set by the rising frequency setting section 752 and a voltage amplitude set by the voltage amplitude setting section 753.

The pump control portion 756 outputs a drive signal to the liquid feeding pump 20 so as to drive the liquid feeding pump 20. The momentum display control portion 757 performs control of displaying a momentum indication value (that is, the present value of the momentum P) allocated to a currently selected lever position of the momentum adjustment lever 713 on the display unit 73.

The storage unit 77 is implemented by various integrated circuit (IC) memories such as a read only memory (ROM), a flash ROM, or a random access memory (RAM), or a storage medium such as a hard disk. The storage unit 77 stores in advance a program for operating the liquid ejection system 1 and thus realizing various functions of the liquid ejection system 1, data used during execution of the program, and the like, or temporarily stores data whenever a process is performed.

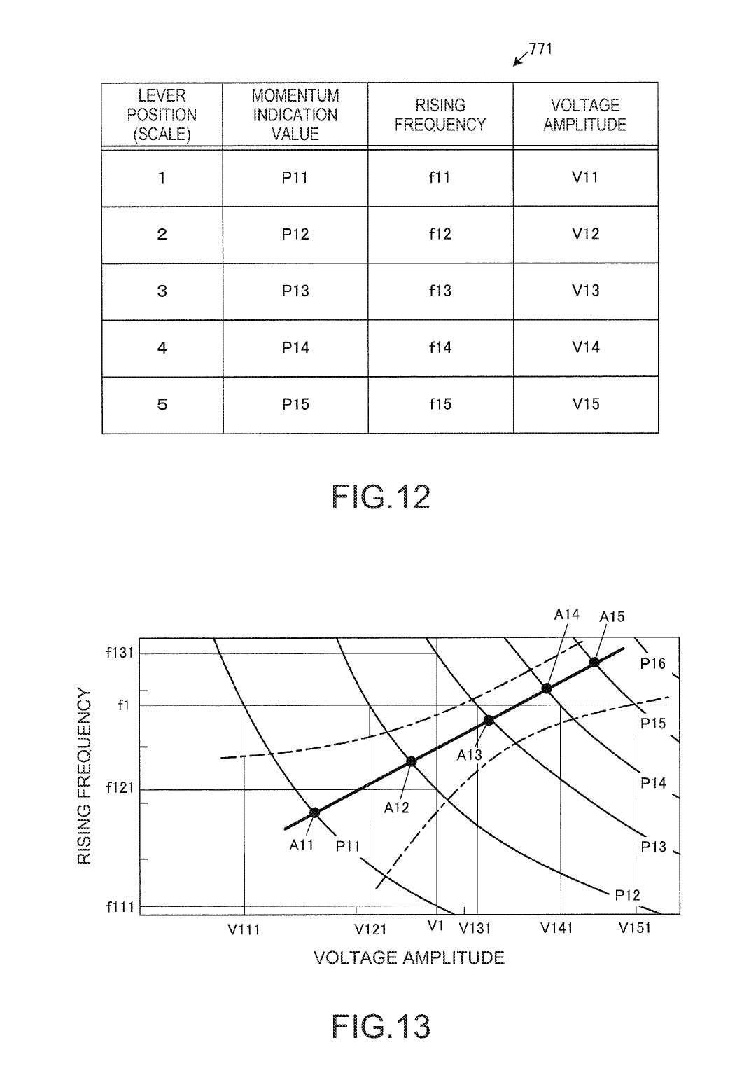

The storage unit 77 stores a momentum conversion table 771. The momentum conversion table 771 is a data table, described with reference to FIG. 9, in which correspondence relationships among the momentum P, the rising frequency, and the voltage amplitude are set, and an example thereof is illustrated in FIG. 12.

FIG. 12 is a diagram illustrating a data configuration example of the momentum conversion table 771. As illustrated in FIG. 12, in the momentum conversion table 771, a momentum indication value allocated to a corresponding lever position, a rising frequency, and a voltage amplitude are set in correlation with the lever position (scale). In a case where the momentum adjustment lever 713 is operated, the rising frequency setting section 752 reads a rising frequency at a selected lever position from the momentum conversion table 771, and sets the rising frequency. The voltage amplitude setting section 753 reads a voltage amplitude at the selected lever position from the momentum conversion table 771, and sets the voltage amplitude. Consequently, for example, in a case where a lever position of the momentum adjustment lever 713 is set to the scale of “3”, the piezoelectric element 45 is controlled with a drive voltage waveform which causes the momentum P to be the momentum indication value P13 allocated to the lever position “3” of the momentum adjustment lever 713.

The data configuration example of the momentum conversion table 771 illustrated in FIG. 12 is only an example, and, as described above, in addition to the straight line or the curve indicated by the bold line or the dot chain line in FIG. 9, desired straight lines or curves may be used as a reference line. In other words, rising frequencies and voltage amplitudes at intersections between the desired reference line and respective contour lines of the momentum indication values P11, P12 . . . may be set in the momentum conversion table 771.

The momentum conversion table 771 may be set to obtain the momentum indication values P11, P12 . . . by making the voltage amplitude constant and changing the rising frequency. For example, as illustrated in FIG. 13, the voltage amplitude may be set to be constant as V1, and rising frequencies which are rising frequencies f111, f121, . . . causing the momentum indication values P11, P12, . . . may be set in the momentum conversion table 771. The momentum P may be changed and set to an indicated value by changing and setting a rising frequency on the basis of the momentum conversion table 771.

The momentum conversion table 771 may be set to obtain the momentum indication values P11, P12 . . . by making the rising frequency constant and changing the voltage amplitude. For example, as illustrated in FIG. 13, the rising frequency may be set to be constant as f1, and voltage amplitudes which are voltage amplitudes V111, V121, . . . causing the momentum indication values P11, P12, . . . may be set in the momentum conversion table 771. The momentum P may be changed and set to an indicated value by changing and setting a voltage amplitude on the basis of the momentum conversion table 771.

A correspondence relationship between the momentum P, the rising frequency, and the voltage amplitude in FIG. 13 is the same as that in FIG. 9.

[Flow of Process]

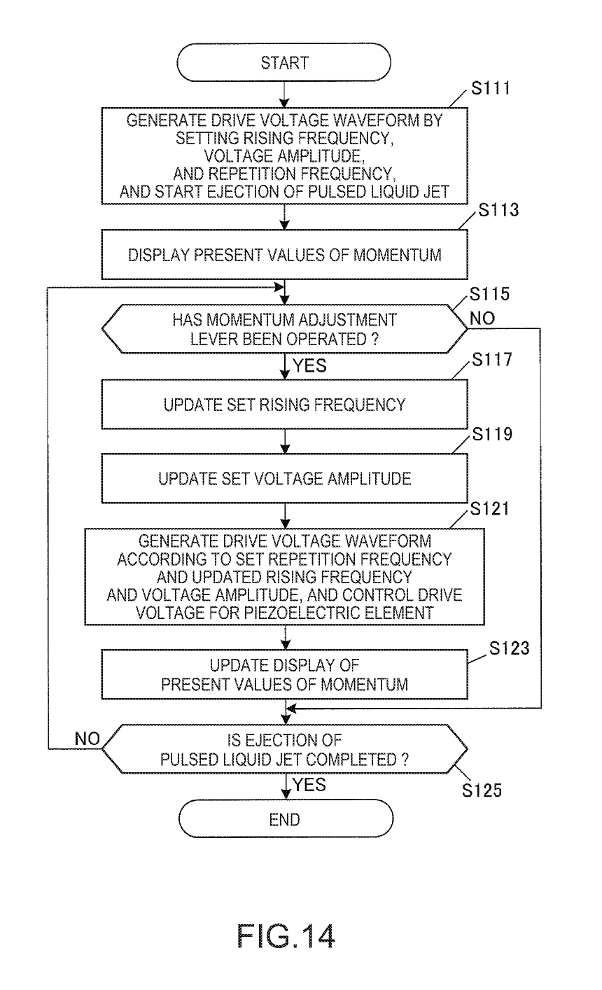

FIG. 14 is a flowchart illustrating a flow of a process performed by the control unit 75 when a pulsed liquid jet is ejected. As illustrated in FIG. 14, if the power button 711 is operated so that power is supplied to the liquid ejection control apparatus 70-1, and thus an instruction for starting of ejection of a pulsed liquid jet is given by the ejection switch 715, the pump control portion 756 drives the liquid feeding pump 20, and the piezoelectric element control portion 751 drives the piezoelectric element 45 so as to start ejection of the pulsed liquid jet (step S111). At this time, the rising frequency setting section 752 acquires a currently selected lever position of the momentum adjustment lever 713, reads a rising frequency from the momentum conversion table 771, and sets the rising frequency. The voltage amplitude setting section 753 reads a voltage amplitude at the acquired lever position from the momentum conversion table 771, and sets the voltage amplitude. The repetition frequency setting section 754 acquires a currently selected lever position of the repetition frequency setting lever 714, and sets a repetition frequency. The piezoelectric element control portion 751 generates a drive voltage waveform according to the set rising frequency, voltage amplitude, and repetition frequency, and applies a drive signal having the generated drive voltage waveform to the piezoelectric element 45.

The momentum display control portion 757 performs control of reading a momentum indication value allocated to the acquired lever position from the momentum conversion table 771, and displaying the read momentum indication value on the display unit 73 (step S113).

Thereafter, the control unit 75 monitors an operation on the momentum adjustment lever 713 in step S115 until it is determined that ejection of a pulsed liquid jet is finished through an operation on the ejection switch 715 (NO in step S125). In a case where the momentum adjustment lever 713 is operated (YES in step S115), the rising frequency setting section 752 reads a rising frequency at a selected lever position from the momentum conversion table 771, and updates the set rising frequency (step S117), and the voltage amplitude setting section 753 reads a voltage amplitude at the selected lever position from the momentum conversion table 771, and updates the set voltage amplitude (step S119). Next, the piezoelectric element control portion 751 generates a drive voltage waveform according to the set repetition frequency, rising frequency, and voltage amplitude, and applies a drive signal having the generated drive voltage waveform to the piezoelectric element 45 (step S121).

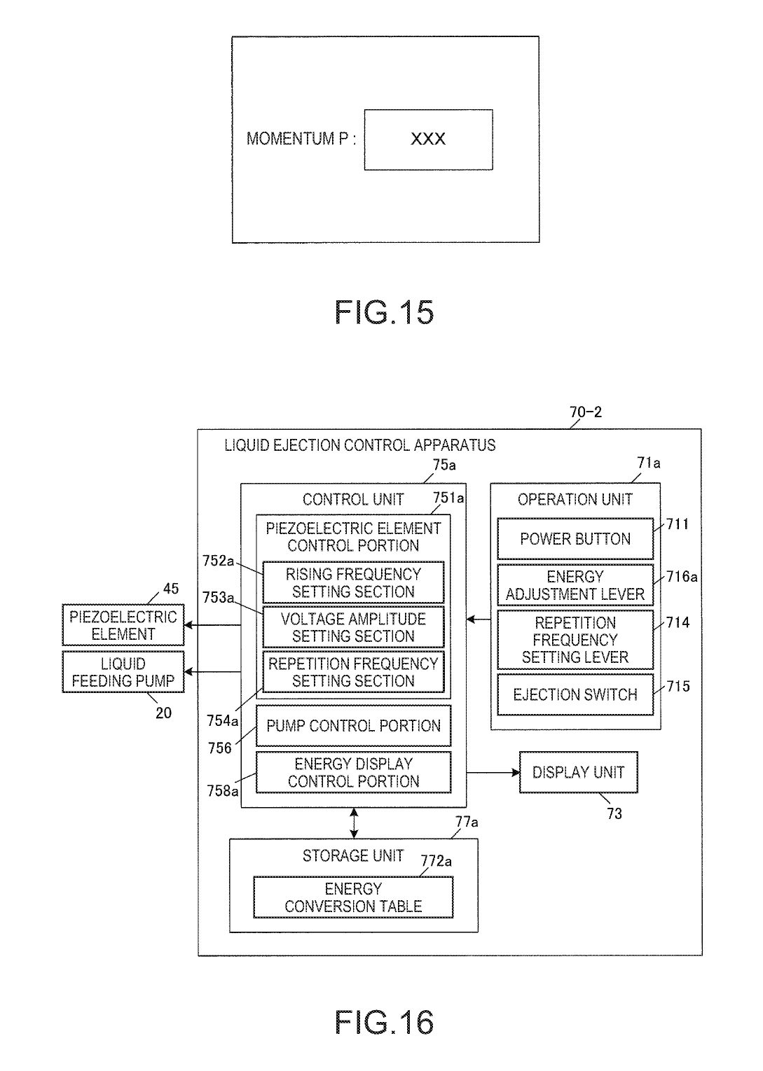

The momentum display control portion 757 reads a momentum indication value allocated to the selected lever position from the momentum conversion table 771, and performs control of updating the display of the display unit 73 (step S123). FIG. 15 is a diagram illustrating a display screen example which is displayed in step S113 and is updated and displayed in step S123. The operator can perform work on the basis of the display screen during surgery while recognizing the present value of the momentum P related to a pulsed liquid jet ejected from the liquid ejection opening 61. Display of a momentum indication value is not limited to the display in numerical values illustrated in FIG. 15, and a value may be displayed in a meter form, or a change in the momentum P due to a changing operation from starting of ejection of a pulsed liquid jet may be displayed in a graph form.

The momentum display control portion 757 may not only display an indication value of the momentum P but may also display the indication value of the momentum P and a repetition frequency together on the display unit 73. In addition to the indication value of the momentum P and the repetition frequency, at least one of the present rising frequency (or rising time) and a voltage amplitude may also be displayed.

According to Example 1, it is possible to control a drive voltage waveform for the piezoelectric element 45 according to a rising frequency and a voltage amplitude which are optimal for achieving a cut depth and a cut volume corresponding to an operation sense on the basis of preset correspondence relationships among the momentum P, a rising frequency, and a voltage amplitude. For example, since the momentum P is changed by an amount corresponding to a scale interval if the momentum adjustment lever 713 is moved by one scale, it is possible to set a cut depth or a cut volume to be suitable for a user's sense or intention and thus to improve convenience.

### Example 2

Next, Example 2 will be described. The same constituent elements as in Example 1 are given the same reference numerals. FIG. 16 is a block diagram illustrating a functional configuration example of a liquid ejection control apparatus in Example 2. As illustrated in FIG. 16, a liquid ejection control apparatus 70-2 includes an operation unit 71a, a display unit 73, a control unit 75a, and a storage unit 77a.

The operation unit 71a includes an energy adjustment lever 716a implemented by the lever switch 813 illustrated in FIG. 1. The energy adjustment lever 716a is used to input an energy indication value. The operator operates the energy adjustment lever 716a, that is, the lever switch 813 illustrated in FIG. 1, so as to select the lever positions with scales such as “1” to “5”, and thus performs an operation of changing the energy E in five steps. The energy indication values are allocated to the respective lever positions in advance so that the energy is increased by a predetermined level in proportion to a numerical value of a corresponding scale. The number of steps of the lever positions is not limited to five steps, and may be set as appropriate, for example, three steps such as “large”, “intermediate”, and “small”.

The control unit 75a includes a piezoelectric element control portion 751a, a pump control portion 756, and an energy display control portion 758a.

The piezoelectric element control portion 751a includes a frequency setting section 752a which sets a rising frequency according to a lever position of the energy adjustment lever 716a, a voltage amplitude setting section 753a which sets a voltage amplitude according to a lever position of the energy adjustment lever 716a, and a repetition frequency setting section 754a which sets a repetition frequency according to a lever position of the repetition frequency setting lever 714. The piezoelectric element control portion 751a generates a drive voltage waveform, applies a drive signal having the generated waveform to the piezoelectric element 45, and, at this time, generates the drive voltage waveform according to a rising frequency set by the frequency setting section 752a and a voltage amplitude set by the voltage amplitude setting section 753a.

The energy display control portion 758a performs control of displaying an energy indication value allocated to a currently selected lever position of the energy adjustment lever 716a, on the display unit 73.

The energy display control portion 758a illustrated in FIG. 19 may display not only an indication value of the energy E but also a repetition frequency on the display unit 73. At least one of the present rising frequency (or rising time) and a voltage amplitude may also be displayed.

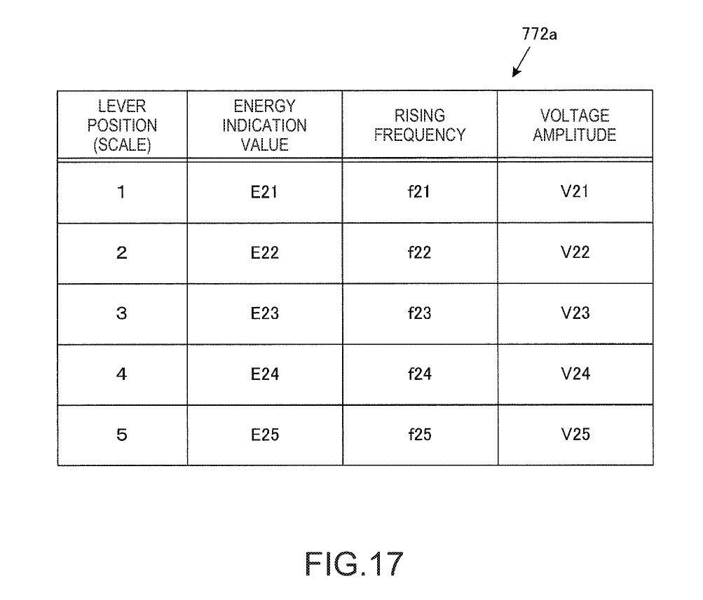

The storage unit 77a stores an energy conversion table 772a. The energy conversion table 772a is a data table, described with reference to FIG. 10, in which correspondence relationships among the energy E, the rising frequency, and the voltage amplitude are set, and an example thereof is illustrated in FIG. 17.

FIG. 17 is a diagram illustrating a data configuration example of the energy conversion table 772a. As illustrated in FIG. 17, in the energy conversion table 772a, an energy indication value allocated to a corresponding lever position, a rising frequency, and a voltage amplitude are set in correlation with the lever position (scale). In a case where the energy adjustment lever 716a is operated, the frequency setting section 752a reads a rising frequency at a selected lever position from the energy conversion table 772a, and sets the rising frequency. The voltage amplitude setting section 753a reads a voltage amplitude at the selected lever position from the energy conversion table 772a, and sets the voltage amplitude. Consequently, for example, in a case where a lever position of the energy adjustment lever 716a is set to the scale of “3”, the piezoelectric element 45 is controlled with a drive voltage waveform which causes the energy E to be the energy indication value E23 allocated to the lever position “3” of the energy adjustment lever 716a.

The data configuration example of the energy conversion table 772a illustrated in FIG. 17 is only an example, and, as described above, in addition to the straight line or the curve indicated by the bold line or the dot chain line in FIG. 10, desired straight lines or curves may be used as a reference line. In other words, rising frequencies and voltage amplitudes at intersections between the desired reference line and respective contour lines of the energy indication values E21, E22 . . . may be set in the energy conversion table 772a.

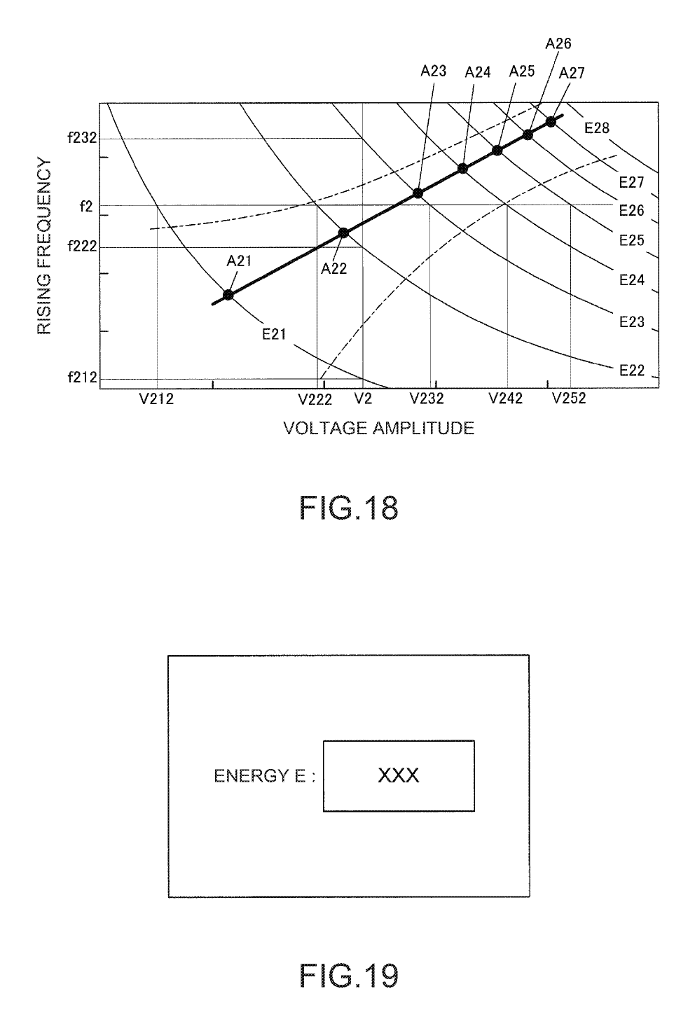

The energy conversion table 772a may be set to obtain the energy indication values E21, E22 . . . by making the voltage amplitude constant and changing the rising frequency, and, conversely, the energy conversion table 772a may be set to obtain the energy indication values E21, E22 . . . by making the rising frequency constant and changing the voltage amplitude. Specifically, for example, as illustrated in FIG. 18, the voltage amplitude may be set to be constant as V2, and rising frequencies which are rising frequencies f212, f222, . . . causing the energy indication values E21, E22 . . . may be set in the energy conversion table 772a, and the rising frequency may be set to be constant as f2, and voltage amplitudes which are voltage amplitudes V212, V222, . . . causing the energy indication values E21, E22 . . . may be set in the energy conversion table 772a.

A correspondence relationship between the energy E, the rising frequency, and the voltage amplitude in FIG. 18 is the same as that in FIG. 10.

According to Example 2, it is possible to control a drive voltage waveform for the piezoelectric element 45 according to a rising frequency and a voltage amplitude which are optimal for achieving a cut depth and a cut volume corresponding to an operation sense on the basis of preset correspondence relationships among the energy E, a rising frequency, and a voltage amplitude. For example, since the energy E is changed by an amount corresponding to a scale interval if the energy adjustment lever 716a is moved by one scale, it is possible to set a cut depth or a cut volume to be suitable for a user's sense or intention and thus to improve convenience.

In the above-described embodiment, a description has been made of a case of performing an operation of changing the momentum P in steps by using the lever switch 813 (momentum adjustment lever 713) or a case of performing an operation of changing energy E in steps by using the lever switch 813 (energy adjustment lever 716a). In contrast, the lever switch 813 has a configuration in which a momentum indication value or an energy indication value can be adjusted steplessly among lever positions with scales.

Regarding a specific process, for example, in a case of the momentum P, if a lever position between scales is selected, momentum indication values, rising frequencies, and voltage amplitudes correlated with lever positions before and after selected momentum P are read by referring to the momentum conversion table 771 illustrated in FIG. 12. The read rising frequencies and voltage amplitudes are linearly interpolated, and thus a rising frequency and a voltage amplitude corresponding to the currently selected momentum P are specified.

In addition to rising frequencies and voltage amplitudes before and after the selected momentum P, rising frequencies and voltage amplitudes corresponding to lever positions (momentum indication values) before the previous scale and after the following scale may be read so as to undergone polynomial interpolation, and thus a rising frequency and a voltage amplitude corresponding to the currently selected momentum P may be specified.

Alternatively, the reference line indicated by the bold line or the dot chain line illustrated in FIG. 9 is actually expressed as a curve in a space having a voltage amplitude, a rising frequency, and a momentum as coordinate axes. An equation corresponding to the curve may be obtained in advance, and a rising frequency and a voltage amplitude corresponding to the momentum P at a selected lever position may be specified by using the equation.

For example, in a case of the energy E, if a lever position between scales is selected, rising frequencies and voltage amplitudes corresponding to lever positions (energy indication values) of scales before and after selected energy E are read by referring to the energy conversion table 772a illustrated in FIG. 17. The read rising frequencies and voltage amplitudes are linearly interpolated, and thus a rising frequency and a voltage amplitude corresponding to the currently selected energy E are specified.

In addition to rising frequencies and voltage amplitudes before and after the selected energy E, rising frequencies and voltage amplitudes corresponding to lever positions (energy indication values) before the previous scale and after the following scale may be read so as to undergone polynomial interpolation, and thus a rising frequency and a voltage amplitude corresponding to the currently selected energy E may be specified.

Alternatively, the reference line indicated by the bold line or the dot chain line illustrated in FIG. 10 is actually expressed as a curve in a space having a voltage amplitude, a rising frequency, and a momentum as coordinate axes. An equation corresponding to the curve may be obtained in advance, and a rising frequency and a voltage amplitude corresponding to the energy E at a selected lever position may be specified by using the equation.

The momentum conversion tables 771 and the energy conversion tables 772a may be prepared and be stored in the storage units 77 and 77a for respective types of liquid ejection devices 30, and the momentum conversion table 771 or the energy conversion table 772a corresponding to the type of liquid ejection device 30 may be selectively used. For example, it is preferable to prepare tables for respective types of liquid ejection device 30 in which structures related to ejection of a pulsed liquid jet are different from each other, such as differences in inner diameters and lengths of the liquid ejection opening 61 and the nozzle 60, differences in an inner diameter and a length of the ejection tube 50, differences in characteristics of the piezoelectric element 45, and a difference in a volume of the pressure chamber 44. This is because the liquid ejection device 30 may be replaced with a different type thereof depending on a cutting target object, for example, an affected part in a case where the liquid ejection system is used for a surgery application, depending on the type of food in a case where the liquid ejection system is used for a food processing application, depending on the kind of each material in a case where the liquid ejection system is used for a cutting processing application of a gel material, or a resin material such as rubber or plastic, that is, depending on a shape density, a destruction threshold value, elasticity and viscosity of a target material to be cut and processed.

More preferably, information indicating the type of liquid ejection device 30 is stored in the liquid ejection device, and the liquid ejection control apparatus 70 reads the information from the liquid ejection device 30 connected thereto, and automatically switch between the momentum conversion tables 771 or the energy conversion tables 772a.

In the above-described embodiment, a rising frequency has been exemplified as an index value related to rising. In contrast, the rising time Tpr may be used instead of the rising frequency.

The momentum adjustment lever 713 or the energy adjustment levers 716a is not limited to case of being implemented by the lever switch 813, and may be implemented by, for example, a dial switch or a button switch. The levers may be implemented by a key switch based on software in a case where the display unit 73 is a touch panel. In this case, a user performs a touch operation on the touch panel as the display unit 73 so as to perform an operation of inputting an indication value of momentum or energy.

In the above-described embodiment, a description has been made of a case where the piezoelectric element control portions 751 and 751a generate a drive voltage waveform according to set repetition frequency, rising frequency, and voltage amplitude (for example, step S121 in FIG. 14). A drive voltage waveform corresponding to one cycle may be generated in advance for each combination of the repetition frequency, the rising frequency, and the voltage amplitude, and may be stored in the storage units 77 and 77a as waveform data correlated with the combination. Waveform data corresponding to the combination of the set repetition frequency, rising frequency, and voltage amplitude may be read, and a drive signal based on the read waveform data may be applied to the piezoelectric element 45.

In the above-described embodiment, a description has been made of a configuration in which a pulsed liquid jet is ejected within a range in which the momentum is equal to or more than 2 nNs and is equal to or less than 2 mNs, and the kinetic energy is equal to or more than 2 nJ and is equal to or less than 200 mJ, but, more preferably, a pulsed liquid jet is ejected within a range in which the momentum is equal to or more than 20 nNs and is equal to or less than 200 μNs, and the kinetic energy is equal to or more than 40 nJ and is equal to or less than 10 mJ. With this configuration, it is possible to appropriately cut a living tissue or a gel material.

### REFERENCE SIGNS LIST

1 LIQUID EJECTION SYSTEM
10 CONTAINER
20 LIQUID FEEDING PUMP
30 LIQUID EJECTION DEVICE
40 PULSE FLOW GENERATOR
44 PRESSURE CHAMBER
45 PIEZOELECTRIC ELEMENT
46 DIAPHRAGM
50 EJECTION TUBE
60 NOZZLE
61 LIQUID EJECTION OPENING
70, 70-1, AND 70-2 LIQUID EJECTION CONTROL APPARATUS
71 AND 71a OPERATION UNIT
713 MOMENTUM ADJUSTMENT LEVER
716a ENERGY ADJUSTMENT LEVER
73 DISPLAY UNIT
75 AND 75a CONTROL UNIT
751 AND 751a PIEZOELECTRIC ELEMENT CONTROL PORTION
752 AND 752a FREQUENCY SETTING PORTION
753 AND 753a VOLTAGE AMPLITUDE SETTING PORTION
756 PUMP CONTROL PORTION
757 MOMENTUM DISPLAY CONTROL PORTION
758a ENERGY DISPLAY CONTROL PORTION
77 AND 77a STORAGE UNIT
771 MOMENTUM CONVERSION TABLE
772a ENERGY CONVERSION TABLE

1. A liquid ejection control apparatus electrically connected to and controlling a liquid ejection device which ejects a liquid in a pulse form by using a piezoelectric element, the liquid ejection control apparatus comprising:
an operation unit that is used to input an indication value of momentum or kinetic energy related to a pulsed liquid jet ejected from the liquid ejection device; and
a control unit that controls an index value related to an amplitude of a drive voltage waveform applied to the piezoelectric element and an index value of rising of the drive voltage waveform, so that the indication value is obtained.

2. A liquid ejection control apparatus electrically connected to and controlling a liquid ejection device which ejects a liquid in a pulse form by using a piezoelectric element, the liquid ejection control apparatus comprising:
an operation unit that is used to input an indication value of momentum or kinetic energy related to a pulsed liquid jet ejected from the liquid ejection device; and
a control unit that controls an index value related to an amplitude of a drive voltage waveform applied to the piezoelectric element or an index value of rising of the drive voltage waveform, so that the indication value is obtained.

3. The liquid ejection control apparatus according to claim 1, further comprising:
a display control unit that performs control of displaying the indication value of momentum and kinetic energy related to the pulsed liquid jet.

4. The liquid ejection control apparatus according to claim 1,
wherein the liquid ejection device is controlled so that momentum of the pulsed liquid jet is equal to or more than 2 nanonewton seconds (nNs) and is equal to or less than 2 millinewton seconds (mNs), or kinetic energy of the pulsed liquid jet is equal to or more than 2 nanojoules (nJ) and is equal to or less than 200 millijoules (mJ).

5. (canceled)

6. The liquid ejection control apparatus according to any claim 1,
wherein the index value related to the rising is represented by time or a frequency related to the rising of the drive voltage waveform.

7. A liquid ejection system comprising:
a liquid ejection device;
a liquid feeding pump device; and
a liquid ejection control apparatus electrically connected to and controlling the liquid ejection device,
wherein the liquid election control apparatus comprising:
an operation unit that is used to input an indication value of momentum or kinetic energy related to a pulsed liquid jet ejected from the liquid ejection device; and
a control unit that controls an index value related to an amplitude of a drive voltage waveform applied to the piezoelectric element or an index value of rising of the drive voltage waveform, so that the indication value is obtained.

8. (canceled)

9. (canceled)

10. The liquid ejection control apparatus according to claim 2, further comprising:
a display control unit that performs control of displaying the indication value of momentum and kinetic energy related to the pulsed liquid jet.

11. The liquid ejection control apparatus according to claim 2,
wherein the liquid ejection device is controlled so that momentum of the pulsed liquid jet is equal to or more than 2 nanonewton seconds (nNs) and is equal to or less than 2 millinewton seconds (mNs), or kinetic energy of the pulsed liquid jet is equal to or more than 2 nanojoules (nJ) and is equal to or less than 200 millijoules (mJ).

12. The liquid ejection control apparatus according to claim 2,
wherein the index value related to the rising is represented by time or a frequency related to the rising of the drive voltage waveform.
